(19) United States
(12) Patent Application Publication
(10) Pub. No.: US 2026/0354967 A1
(43) Pub. Date: Nov. 12, 2026

(54) System and Method for Producing Custom Silicone Facial Prosthetics via AI-Based 3D Scanning and Additive Manufacturing

(71) Applicant: James E McCord, JR., Lakewood, CO (US)

(72) Inventor: James E McCord, JR., Lakewood, CO (US)

(73) Assignee: James E McCord, Jr., Lakewood, CO (US)

(21) Appl. No.: 19/203,011

(22) Filed: May 8, 2025

(51) Int. Cl. A61F 2/18; B33Y 50/00; G06V 20/64; G06V 40/16

(52) U.S. Cl. CPC A61F 2/186; B33Y 50/00; G06V 20/64; G06V 40/168; A61F 2240/002

(57) ABSTRACT

A novel system and method for producing custom silicone facial prosthetics directly from high-resolution 3D scans using anatomically-trained artificial intelligence (AI). The AI identifies and replicates micro-anatomical features such as pores, wrinkles, and blemishes, enhancing realism and comfort. Biomechanical simulations are used to ensure natural motion with the face. The resulting prosthetic is 3D printed directly from the enhanced model using medical-grade silicone, eliminating the need for traditional molds or manual sculpting.

### FIELD OF THE INVENTION

This disclosure relates to systems and methods for producing custom silicone facial prosthetics directly from high-resolution three-dimensional scans using anatomically trained artificial intelligence and additive manufacturing.

### BACKGROUND OF THE INVENTION

Conventional processes for producing facial prosthetics may rely on physical impressions, molds, manual refinements, and manual sculpting. Such conventional processes may require multiple steps and can introduce delay, discomfort, and production error.

### SUMMARY OF THE INVENTION

Unlike conventional processes that rely on physical impressions, molds, and manual refinements, this invention introduces a complete digital workflow. AI-trained modeling software automatically enhances incomplete or irregular anatomical scan data, enabling full-featured facial prosthetics even for patients with deformities or trauma. The integration of AI texture mapping and facial movement simulation increases realism and clinical utility, while the elimination of molds reduces production time and error.

### DETAILED DESCRIPTION OF THE INVENTION

A system and method for producing custom silicone facial prosthetics directly from high-resolution three-dimensional scans using anatomically trained artificial intelligence is disclosed.

The artificial intelligence identifies and replicates micro-anatomical features such as pores, wrinkles, scars, and blemishes, enhancing realism and comfort.

Biomechanical simulations are used to ensure natural motion with the face.

The resulting prosthetic is three-dimensionally printed directly from the enhanced model using medical-grade silicone, eliminating the need for traditional molds or manual sculpting.

In one method, a high-resolution three-dimensional scan of a patient's face is acquired.

The scan is processed using artificial intelligence to identify facial surface features including pores, wrinkles, and scars.

Deformities or scan gaps may be corrected using trained anatomical inference models.

Motion of the facial region may be simulated to optimize prosthetic fit and attachment.

A three-dimensional model incorporating the identified facial detail is generated.

The model may be directly printed using biocompatible elastomeric silicone without use of any mold.

The artificial intelligence may use a dataset of human facial scans to predict missing or obscured features.

The prosthetic may include printed micro-textures to simulate realistic skin.

### BRIEF DESCRIPTION OF THE DRAWINGS

FIG. 1 illustrates high-resolution three-dimensional scanning of a patient's face.

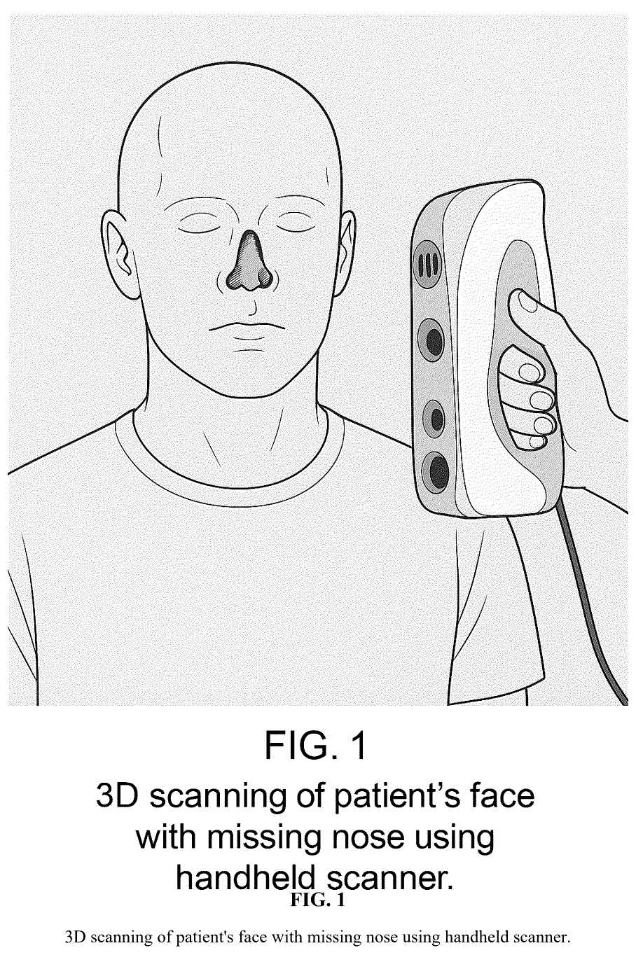

FIG. 2 illustrates an AI-enhanced digital model with skin detail mapping.

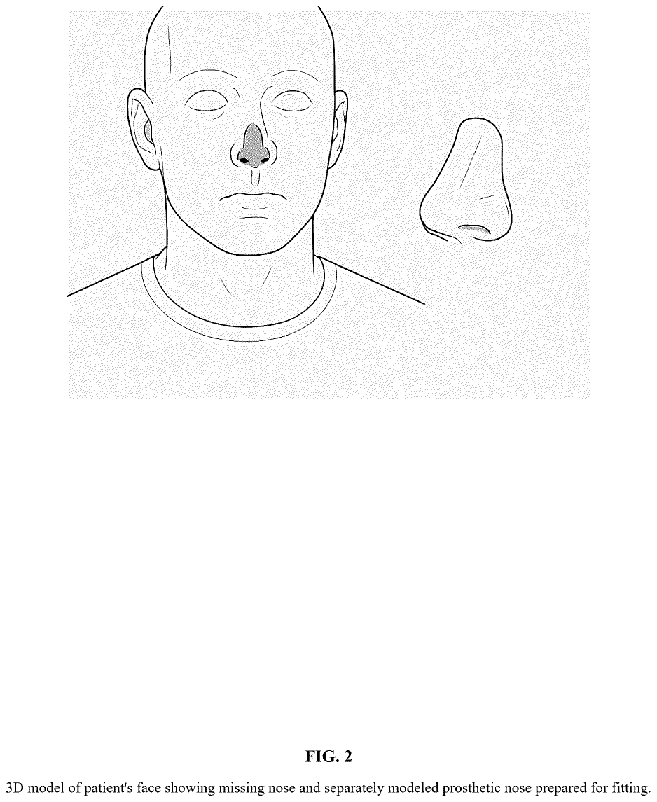

FIG. 3 illustrates a simulation interface showing motion prediction.

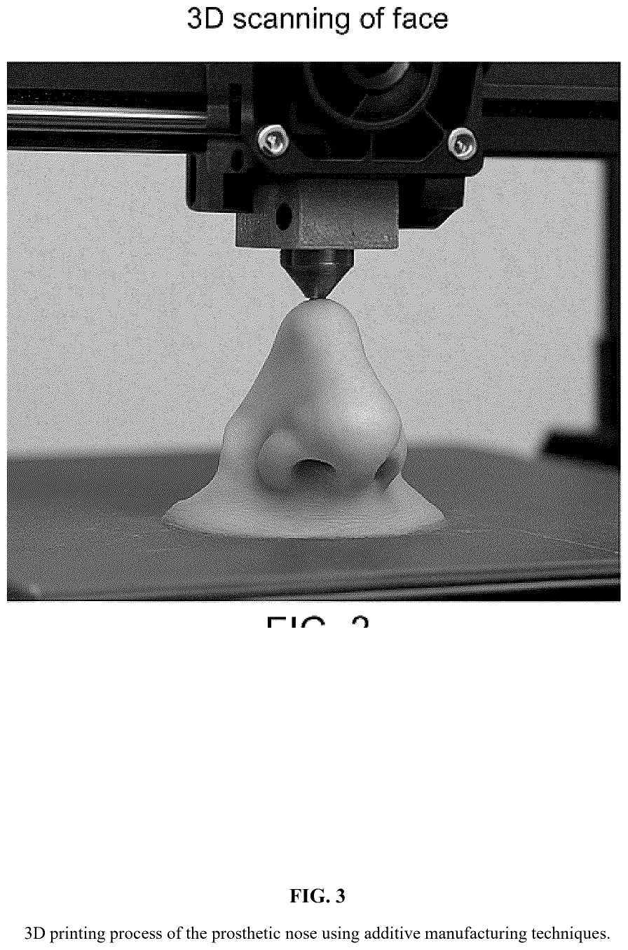

FIG. 4 illustrates a finished silicone prosthetic, three-dimensionally printed without a mold.

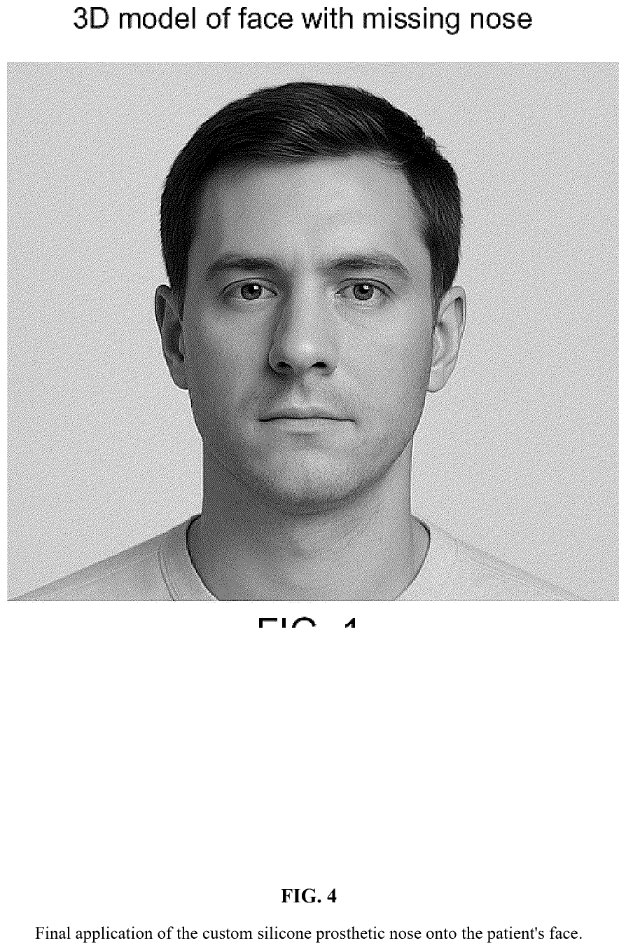

1. A method for producing a silicone facial prosthetic comprising:
(a) acquiring a high-resolution 3D scan of a patient's face;
(b) processing the scan using artificial intelligence to identify facial surface features including pores, wrinkles, and scars;
(c) correcting deformities or scan gaps using trained anatomical inference models;
(d) simulating motion of the facial region to optimize prosthetic fit and attachment;
(e) generating a 3D model incorporating said detail; and
(f) directly printing the model using biocompatible elastomeric silicone without use of any mold.

2. The method of claim 1, wherein said artificial intelligence uses a dataset of human facial scans to predict missing or obscured features.

3. The method of claim 1, wherein said prosthetic includes printed micro-textures to simulate realistic skin.
